(19) United States
(12) Patent Application Publication
(10) Pub. No.: US 2024/0416093 A1
(43) Pub. Date: Dec. 19, 2024

(54) MEDICAL SYSTEM AND FLUID CIRCULATION SYSTEM

(71) Applicant: TERUMO KABUSHIKI KAISHA, Tokyo (JP)

(72) Inventor: Takashi KITAOKA, Hadano city (JP)

(73) Assignee: TERUMO KABUSHIKI KAISHA, Tokyo (JP)

(21) Appl. No.: 18/731,618

(22) Filed: Jun. 3, 2024

(30) Foreign Application Priority Data
Jun. 16, 2023 (JP) 2023-099692

(51) Int. Cl. A61M 27/00; A61M 1/00; A61M 25/00; A61M 39/02

(52) U.S. Cl. CPC A61M 27/006; A61M 1/777; A61M 25/0097; A61M 39/0247; A61M 2025/0031; A61M 2027/004; A61M 2039/0276; A61M 2202/0464; A61M 2210/1003

(57) ABSTRACT

A medical system includes a control unit that executes control of setting a first period in which cerebrospinal fluid is discharged from a body cavity while a fluid is injected into the body cavity and a second period in which the fluid is not injected into the body cavity and the cerebrospinal fluid is not discharged from the body cavity and alternately repeating the first period and the second period.

### CROSS-REFERENCES TO RELATED APPLICATIONS

This application claims priority to Japanese Application No. 2023-099692 filed on Jun. 16, 2023, the entire content of which is incorporated herein by reference.

### TECHNOLOGICAL FIELD

The present disclosure generally relates to a medical system and a fluid circulation system which are used for treatment of a brain disease.

### BACKGROUND DISCUSSION

When a cerebral infarction, for example, occurs as a brain disease, a blood flow supplying oxygen to brain cells is blocked, and the brain cells are damaged. Therefore, when the cerebral infarction occurs, early reperfusion (i.e., restoration) of the blood flow is necessary. As one of treatments for the cerebral infarction, it has been proposed that a high oxygen solution such as oxygenated cerebrospinal fluid is injected into a body cavity in which cerebrospinal fluid of a patient is present, and oxygen is directly supplied to oxygen-deficient brain cells.

International Patent Application Publication No. WO 2021/192539 A discloses a catheter system capable of introducing and drawing a fluid into and from a living body through a single lumen. The catheter system described in International Patent Application Publication No. WO 2021/192539 A includes a catheter, an injection actuator for supplying a fluid to an injection port through a lumen in a tubular body, a suction actuator for drawing in a fluid from a suction port through the lumen in the tubular body, a detector for detecting a state of intracranial pressure, and a controller for controlling the injection actuator and the suction actuator. The suction port of the catheter is in a closed state when the injection port of the catheter is in an open state. In addition, the injection port of the catheter is in a closed state when the suction port of the catheter is in an open state. Then, the controller alternately operates the injection actuator and the suction actuator in order to keep the state of intracranial pressure detected by the detector within a certain range.

Here, in a case where a fluid such as a high oxygen solution is injected into a body cavity for the purpose of treating a brain disease, it is necessary to keep the intracranial pressure within a certain range. A normal range of the intracranial pressure is about 5 mmHg to 15 mmHg. If the pressure from an intracranial space to an intraspinal space fluctuates, there is a possibility that brain damage can occur. For example, when the intracranial pressure increases to 20 mmHg or more, there is a possibility that intracranial hypertension (that is, various symptoms developed by a load on brain tissue) occurs. On the other hand, when the intracranial pressure decreases to 5 mmHg or less, there is a possibility that cerebrospinal fluid hypovolemia (that is, various symptoms developed as a position of the brain tissue cannot be kept) occurs.

The body cavity (for example, a subarachnoid space and cerebral ventricles) in which cerebrospinal fluid is present are substantially closed spaces. Therefore, in order to keep the intracranial pressure within the certain range, it is necessary to keep the amount of the cerebrospinal fluid in the body cavity within a certain range. In order to keep the amount of the cerebrospinal fluid in the body cavity within the certain range, it is required to rapidly discharge the same amount of cerebrospinal fluid as the amount of the fluid injected into the body cavity.

However, for example, in a case where a fluid is circulated by a pump through a catheter for injection and a catheter for discharge, which are inserted into the subarachnoid space, from the vicinity of lumbar vertebrae to inject the fluid into the body cavity, a flow (that is, a locally circulating flow) in which the fluid injected into the body cavity from an injection port of the catheter for injection flows toward a discharge port of the catheter for discharge is formed depending on an injection timing and a discharge timing. This decreases a flow of the fluid from the injection port of the catheter for injection toward a treatment area of the brain decreases, and there is a problem that the fluid cannot be efficiently delivered to the treatment area of the brain.

In order to solve this problem, it is conceivable to dispose the injection port of the catheter for injection in the vicinity of the brain and dispose the discharge port of the catheter for discharge in the vicinity of the lumbar vertebrae, thereby sufficiently securing a distance between the injection port and the discharge port and suppressing an influence of the locally circulating flow as much as possible. However, in a case where an attempt is made to insert the catheter into the vicinity of the brain, the catheter is inserted over a long distance into the curved subarachnoid space around which nerve tissue such as the spinal cord runs. Since such insertion of the catheter into a deep portion of the subarachnoid space involves a risk of damaging the nerve tissue, it is desirable to shorten an insertion length of the catheter as much as possible.

In addition, a circulation circuit of the fluid is desirably a closed system for infection prevention. However, in a case where the circulation circuit of the fluid is the closed system, there is a possibility that air bubbles are generated in the circulation circuit caused by the cavitation phenomenon to increase a total volume of the fluid from a volume in an initial stage of a treatment, or water is lost caused by evaporation in the process of oxygenating the cerebrospinal fluid to decrease the total volume of the fluid from the volume in the initial stage of the treatment. If the total volume of the fluid increases or decreases in the circulation circuit of the closed system, there is a possibility that the intracranial pressure cannot be kept in the certain range.

### SUMMARY

A medical system and a fluid circulation system are disclosed, which are capable of efficiently delivering a fluid to a treatment area of a brain while suppressing fluctuations in intracranial pressure.

The disclosure provides (1) a medical system configured to inject a fluid into a body cavity in which cerebrospinal fluid of a subject is present and discharge the cerebrospinal fluid present in the body cavity from the body cavity, the medical system including a control unit configured to execute control of setting a first period in which the cerebrospinal fluid is discharged from the body cavity while the fluid is injected into the body cavity and a second period in which the fluid is not injected into the body cavity and the cerebrospinal fluid is not discharged from the body cavity and alternately repeating the first period and the second period.

According to the medical system of (1), the control unit is configured to execute control to discharge the cerebrospinal fluid from the body cavity while injecting the fluid into the body cavity in which the cerebrospinal fluid of the subject is present in the first period. In addition, the control unit is configured to execute control not to inject the fluid into the body cavity and not to discharge the cerebrospinal fluid from the body cavity in the second period. Then, the control unit is configured to execute control to alternately repeat the first period and the second period. As a result, the injection of the fluid and the discharge of the cerebrospinal fluid are repeated simultaneously and intermittently. Therefore, there occurs a period in which a locally circulating flow of the fluid injected into the body cavity from an injection port toward a discharge port disappears, and diffusion due to a concentration difference of oxygen or the like contained in the fluid progresses in the period in which the locally circulating flow disappears. As a result, the fluid can be efficiently delivered to a treatment area of a brain. In addition, since the injection of the fluid and the discharge of the cerebrospinal fluid are simultaneously performed, the amount of the cerebrospinal fluid in the body cavity can be kept in a certain range, which can suppress fluctuations in intracranial pressure.

(2) In the medical system of (1), it is preferable that an injection amount of the fluid in the first period is substantially identical to a discharge amount of the cerebrospinal fluid in the first period.

According to the medical system of (2), since the injection amount of the fluid is substantially identical to the discharge amount of the cerebrospinal fluid, the amount of the cerebrospinal fluid in the body cavity can be more reliably kept in the certain range, which makes it possible to more reliably suppress the fluctuations in the intracranial pressure.

(3) In the medical system of (1) or (2), it is preferable that a cycle from the start of the first period to the end of the second period is substantially constant.

According to the medical system of (3), movement of the fluid and the diffusion due to the concentration difference of oxygen or the like contained in the fluid stably progress. As a result, the fluid can be efficiently delivered to a treatment area of a brain. In addition, the amount of the cerebrospinal fluid in the body cavity can be stably kept in the certain range, which can suppress fluctuations in intracranial pressure.

(4) In the medical system according to any one of (1) to (3), it is preferable that the duration of the second period is 15 seconds or more.

According to the medical system of (4), it is possible to secure a sufficient time for the progress of the movement of the fluid and the diffusion due to the concentration difference of oxygen or the like contained in the fluid, and the locally circulating flow remaining by inertia is also stopped. As a result, the fluid can be efficiently delivered to a treatment area of a brain.

(5) In the medical system according to any one of (1) to (4), it is preferable that the control is a first control, and the control unit is further is configured to execute a second control of setting a third period in which the fluid is injected into the body cavity and the cerebrospinal fluid is not discharged from the body cavity and a fourth period in which the fluid is not injected into the body cavity and the cerebrospinal fluid is discharged from the body cavity, alternately repeating the third period and the fourth period, and to set an injection amount of the fluid in the third period to be substantially identical to a discharge amount of the cerebrospinal fluid in the fourth period, and select and execute any one of the first control and the second control.

According to the medical system of (5), the control unit is configured to inject the fluid into the body cavity and does not discharge the cerebrospinal fluid from the body cavity in the third period. In addition, the control unit does not inject the fluid into the body cavity and discharges the cerebrospinal fluid from the body cavity in the fourth period. Then, the control unit alternately repeats the third period and the fourth period to make an injection amount of the fluid substantially identical to a discharge amount of the cerebrospinal fluid. As a result, the injection of the fluid and the discharge of the cerebrospinal fluid are alternately repeated in the same amount in the second control as long as an allowable fluctuation range of the intracranial pressure is not exceeded. Therefore, an injection period of the fluid and a discharge period of the cerebrospinal fluid are set at different times, and thus, both the periods are not overlapped, and it is possible to suppress the generation of the locally circulating flow in which the fluid injected into the body cavity from the injection port flows toward the discharge port. Furthermore, the control unit can select and execute any one of the control of (1) (that is, the first control) and the control of (2) (that is, the second control). As a result, the medical system of (5) can flexibly cope with various situations such as the intracranial pressure of the subject and the amount of the cerebrospinal fluid, and can efficiently deliver the fluid to the treatment area of the brain.

(6) It is preferable that the medical system of (1) further includes: an injection catheter configured to inject the fluid into the body cavity; and a discharge catheter configured to discharge the cerebrospinal fluid from the body cavity, and a distance between a distal end of the injection catheter and a distal end of the discharge catheter is 30 cm or less at a start of execution of the control.

According to the medical system of (6), since the injection of the fluid and the discharge of the cerebrospinal fluid are repeated simultaneously and intermittently, it is possible to secure a period in which the locally circulating flow in which the fluid injected into the body cavity from the injection port of the injection catheter flows toward the discharge port of the discharge catheter disappears even if the distance between the distal end of the injection catheter and the distal end of the discharge catheter is 30 cm or less at the start of execution of the control. As a result, the diffusion due to the concentration difference of oxygen or the like contained in the fluid progresses in the period in which the locally circulating flow disappears. As a result, the fluid can be efficiently delivered to the treatment area of the brain without deeply inserting the injection catheter into a living body.

The disclosure provides (7) a fluid circulation system configured to inject a fluid into a body and discharge the fluid out of the body to circulate the fluid, the fluid circulation system including: an injection catheter configured to inject the fluid into the body; a discharge catheter configured to discharge the fluid from the inside of the body to the outside of the body; a system circuit connected to the injection catheter and the discharge catheter; an injection fluid storage unit configured to store the fluid supplied from the system circuit and injected into the body; a discharge fluid storage unit configured to store the fluid discharged from the inside of the body to the outside of the body; a first fluid delivery unit that is connected to the injection fluid storage unit and the injection catheter, the first fluid delivery unit configured to supply the fluid to the injection fluid storage unit, and supply the fluid stored in the injection fluid storage unit to the injection catheter; a second fluid delivery unit that is connected to the discharge fluid storage unit and the discharge catheter, the second fluid delivery unit configured to supply the fluid to the discharge fluid storage unit, and supply the fluid stored in the discharge fluid storage unit to the system circuit; a first flow path switching unit that is provided on a downstream side of the injection fluid storage unit and is configured to switch between a flow path in which the system circuit is connected to the injection fluid storage unit and a flow path in which the injection fluid storage unit is connected to the injection catheter; and a second flow path switching unit that is provided on an upstream side of the discharge fluid storage unit and is configured to switch between a flow path in which the discharge catheter is connected to the discharge fluid storage unit and a flow path in which the discharge fluid storage unit is connected to the system circuit.

According to the fluid circulation system of (7), a circuit for circulating the fluid is divided into a circuit (that is, a biological circuit) including the injection catheter and the discharge catheter and the system circuit connected to the injection catheter and the discharge catheter. In addition, the first fluid delivery unit is configured to supply the fluid to the injection catheter and the second fluid delivery unit is configured to supply the fluid to the system circuit function as a fluid delivery units shared by the biological circuit and the system circuit. In addition, the first flow path switching unit and the second flow path switching unit can switch the flow paths in which the fluid flows. Since the fluid circulation system has such a configuration, the system circuit can cope with an increase in a total volume of the fluid caused by the cavitation phenomenon and a decrease in the total volume of the fluid caused by evaporation while maintaining a situation on the living body side, and can suppress the increase and decrease in the total volume of the fluid. This can suppress fluctuations in intracranial pressure.

(8) In the fluid circulation system of (7), it is preferable that, as the first fluid delivery unit and the second fluid delivery unit operate in conjunction with each other, a volume of the fluid injected into the body is always equal to a volume of the fluid discharged out of the body.

According to the fluid circulation system of (8), the first fluid delivery unit and the second fluid delivery unit operate in conjunction with each other, and function as one fluid delivery unit shared by the biological circuit and the system circuit. Then, the volume of the fluid injected into the body is always equal to the volume of the fluid discharged out of the body. Therefore, the amount of cerebrospinal fluid in the body can be more reliably kept in a certain range, which makes it possible to more reliably suppress the fluctuations in the intracranial pressure.

(9) It is preferable that the fluid circulation system of (7) further includes a piston drive unit, the injection fluid storage unit is a first syringe configured to store the fluid, the discharge fluid storage unit is a second syringe configured to store the fluid, the first fluid delivery unit is a first piston that is capable of sliding and reciprocating in the first syringe, the second fluid delivery unit is a second piston that is configured to slide and reciprocate in the second syringe, and the first piston and the second piston are connected to each other, and reciprocate by the piston drive unit in a state of being connected to each other.

According to the fluid circulation system of (9), the first piston is configured to slide and reciprocate in the first syringe and the second piston is configured to slide and reciprocate in the second syringe reciprocate by the piston drive unit in a state of being connected to each other. As a result, the first fluid delivery unit and the second fluid delivery unit function as one integrated fluid delivery unit shared by the biological circuit and the system circuit. Therefore, the volume of the fluid injected into the body becomes equal to the volume of the fluid discharged out of the body. Therefore, the amount of the cerebrospinal fluid in the body can be kept in the certain range, which can suppress fluctuations in intracranial pressure.

(10) In the fluid circulation system of (9), it is preferable that, as the first syringe and the second syringe are formed to have identical shape and volume, a volume of the fluid injected into the body is always equal to a volume of the fluid discharged out of the body.

According to the fluid circulation system of (10), in such a fixed displacement pump, the amount of the fluid injected and discharged in one cycle does not fluctuate, and an injection amount and a discharge amount can be reliably made identical as compared with a variable displacement pump. Therefore, the amount of cerebrospinal fluid in the body can be more reliably kept in a certain range, which makes it possible to more reliably suppress the fluctuations in the intracranial pressure.

(11) In the fluid circulation system of (7), it is preferable that the system circuit includes an oxygenator that adds oxygen to the fluid discharged out of the body.

According to the fluid circulation system of (11), it is possible to efficiently deliver the high oxygen solution to the treatment area of the brain while suppressing the fluctuations in the intracranial pressure.

A method is disclosed for injecting a fluid into a body cavity in which cerebrospinal fluid of a subject is present and to discharge the cerebrospinal fluid present in the body cavity from the body cavity, the method comprising: setting a first period in which the cerebrospinal fluid is discharged from the body cavity while the fluid is injected into the body cavity and a second period in which the fluid is not injected into the body cavity and the cerebrospinal fluid is not discharged from the body cavity; and alternately repeating the first period and the second period.

According to the disclosure, it is possible to provide the medical system and the fluid circulation system capable of efficiently delivering the fluid to the treatment area of the brain while suppressing the fluctuations in the intracranial pressure.

### BRIEF DESCRIPTION OF THE DRAWINGS

FIG. 1 is a schematic diagram illustrating an outline of a medical system according to an embodiment of the disclosure.

FIG. 2 is a plan view illustrating the vicinity of an injection port of an injection catheter of the embodiment.

FIG. 3 is a plan view illustrating the vicinity of a discharge port of a discharge catheter of the embodiment.

FIG. 4 is a cross-sectional view taken along a cutting plane B-B illustrated in FIG. 3.

FIG. 5 is a schematic view illustrating a flow of a fluid generated in a body cavity immediately after the start of fluid circulation.

FIG. 6 is a schematic view illustrating a flow of the fluid generated in the body cavity during the fluid circulation.

FIG. 7 is a schematic view illustrating a flow of the fluid generated in the body cavity after the stop of the fluid circulation.

FIG. 8 is a graph showing a relationship among an injection amount, a discharge amount, and a fluid amount in the body cavity under control of the embodiment.

FIG. 9 is a schematic view illustrating a flow of the fluid generated in the body cavity in the control of the embodiment.

FIG. 10 is a graph showing a relationship among an injection amount, a discharge amount, and a fluid amount in the body cavity under another control of the embodiment.

FIG. 11 is a schematic view illustrating a flow of the fluid generated in the body cavity by injection in another control of the embodiment.

FIG. 12 is a schematic view illustrating a flow of the fluid generated in the body cavity during the stop of another control of the embodiment.

FIG. 13 is a schematic view illustrating a flow of the fluid generated in the body cavity by discharge in another control of the embodiment.

FIG. 14 is a schematic diagram illustrating an outline of an experiment performed by the present inventor.

FIG. 15 is a table showing an example of results of the experiment performed by the present inventor.

FIGS. 16A to 16C are photographs showing states of the experiment performed by the present inventor.

FIG. 17 is a schematic diagram illustrating an outline of a fluid circulation system according to the embodiment.

FIG. 18 is a schematic diagram for describing an operation of the fluid circulation system according to the embodiment.

FIG. 19 is a schematic diagram for describing the operation of the fluid circulation system according to the embodiment.

FIG. 20 is a schematic diagram for describing another operation of the fluid circulation system according to the embodiment.

FIG. 21 is a schematic diagram for describing another operation of the fluid circulation system according to the embodiment.

FIG. 22 is a schematic diagram for describing another operation of the fluid circulation system according to the embodiment.

FIG. 23 is a schematic diagram for describing another operation of the fluid circulation system according to the embodiment.

### DETAILED DESCRIPTION

Set forth below with reference to the accompanying drawings is a detailed description of embodiments of a medical system and a fluid circulation system which are used for treatment of a brain disease.

Note that the embodiment described below is a preferred specific example of the disclosure, and thus various technically preferable limitations are given. However, the scope of the disclosure is not limited to these aspects unless there is a description to limit the disclosure in the following description. In addition, in the drawings, similar components are denoted by the same reference signs, and the detailed description of the similar components denoted by the same reference signs will be appropriately omitted.

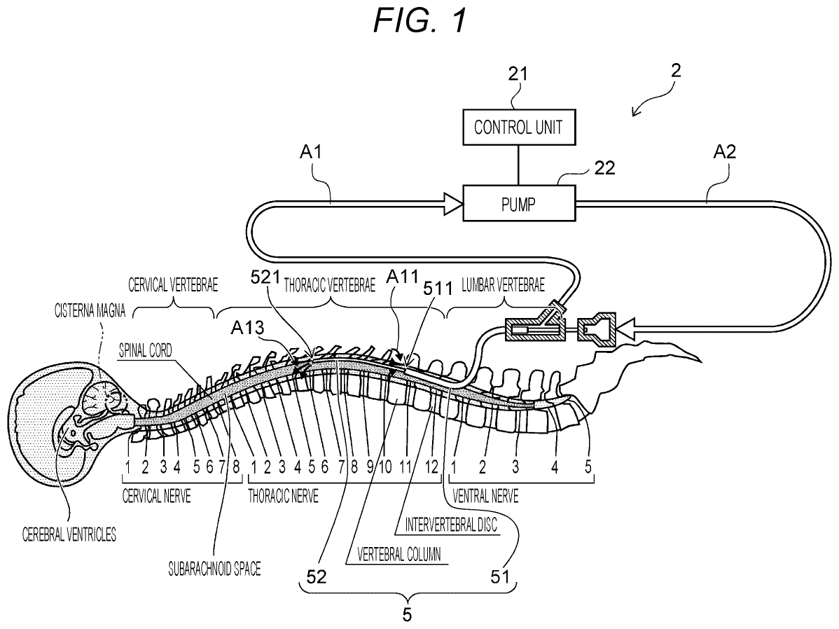

FIG. 1 is a schematic diagram illustrating an outline of a medical system according to the embodiment of the disclosure.

A medical system 2 according to the embodiment injects a fluid into a body cavity in which cerebrospinal fluid (CSF) of a subject is present, and discharges a fluid present in the body cavity from the body cavity. The cerebrospinal fluid is present mainly in a subarachnoid space and cerebral ventricles. That is, the body cavity in which the cerebrospinal fluid is present includes the subarachnoid space and the cerebral ventricles.

As illustrated in FIG. 1, the medical system 2 includes a control unit 21, a pump 22, and a medical device 5. The control unit 21 is connected to the pump 22 and transmits a control signal to the pump 22 to control an operation of the pump 22. The pump 22 operates on the basis of a control signal transmitted from the control unit 21, and draws and discharges the cerebrospinal fluid from the body cavity of the subject through the medical device 5 as indicated by an arrow A1 in FIG. 1. The cerebrospinal fluid discharged out of the body cavity by the pump 22 is generated into a fluid (that is, a high oxygen solution) having an oxygen concentration higher than a normal oxygen concentration of cerebrospinal fluid by, for example, an oxygenation mechanism or the like. Then, the pump 22 injects the fluid into the body cavity through the medical device 5 as indicated by an arrow A2 illustrated in FIG. 1.

Note that the fluid to be injected into the body cavity is not limited to the high oxygen solution. For example, the fluid to be injected into the body cavity may be a fluid containing a drug and obtained by adding the drug to the cerebrospinal fluid during extracorporeal circulation, or may be cerebrospinal fluid filtered with a filter to remove an undesirable substance during extracorporeal circulation. In addition, the fluid to be injected into the body cavity may be one obtained by performing certain processing, such as irradiation with energy or heating, on the cerebrospinal fluid and returned into the body cavity. In the following description, an example in which the fluid to be injected into the body cavity is the high oxygen solution may be given for convenience of description. In addition, in an initial stage of a treatment, lactated Ringer's solution can be used as a substitute for the cerebrospinal fluid in the fluid to be injected into the living body. In the embodiment, the cerebrospinal fluid and artificial cerebrospinal fluid such as the lactated Ringer's solution, a mixed solution of the cerebrospinal fluid and the lactated Ringer's solution, and the like may be collectively referred to as the fluid.

As illustrated in FIG. 1, the medical device 5 includes a discharge catheter 51 and an injection catheter 52, and is inserted into the subarachnoid space from the vicinity of lumbar vertebrae in a lateral decubitus state. A distal position where the injection catheter 52 is disposed is desirably between the vicinity of the sixth thoracic vertebra, called T6, from the top and a position of the first lumbar vertebra, called L1, from the top in consideration of safety at the time of insertion. For example, as indicated by an arrow A11 illustrated in FIG. 1, the discharge catheter 51 draws the cerebrospinal fluid present in the body cavity from a discharge port 511 and discharges the cerebrospinal fluid out of the body cavity. For example, as indicated by an arrow A13 illustrated in FIG. 1, the injection catheter 52 injects the fluid from an injection port 521 into the body cavity such as the subarachnoid space where the cerebrospinal fluid is present. In addition, as an example in FIG. 1, the discharge catheter 51 and the injection catheter 52 are inserted from the first lumber vertebra L1 from the top, and the discharge port 511 at the tip of the discharge catheter 51 is positioned near the eleventh thoracic vertebra T11 from the top. However, the insertion positions and the tip positions of the discharge catheter 51 and the injection catheter 52 are not particularly limited as long as the positions ensure safety during insertion.

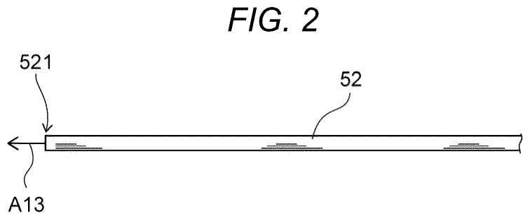

FIG. 2 is a plan view illustrating the vicinity of the injection port of the injection catheter of the embodiment.

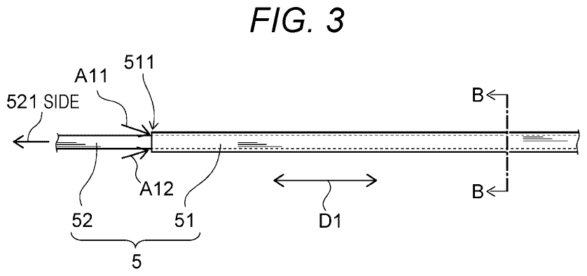

FIG. 3 is a plan view illustrating the vicinity of the discharge port of the discharge catheter of the embodiment.

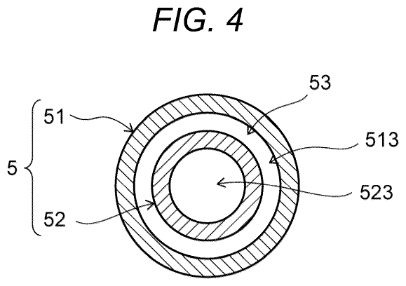

FIG. 4 is a cross-sectional view taken along the cutting plane B-B illustrated in FIG. 3.

As illustrated in FIG. 3, a distal portion of the discharge catheter 51 is opened as the discharge port 511 and is disposed in the subarachnoid space in the vicinity of the lumbar vertebrae. For example, as indicated by arrows A11 and A12 illustrated in FIG. 3, the discharge catheter 51 is inserted into the subarachnoid space of the subject, and draws the cerebrospinal fluid present in the subarachnoid space in the vicinity of the lumbar vertebrae into a space 53 (see FIG. 4) between a lumen 513 of the discharge catheter 51 and an outer surface of the injection catheter 52 through the discharge port 511. Note that a force for drawing the cerebrospinal fluid is given by the pump 22 as described above with respect to FIG. 1. Then, as indicated by the arrow A1 illustrated in FIG. 1, the discharge catheter 51 discharges the cerebrospinal fluid out of the body cavity of the subject through the space 53.

An outer diameter of the injection catheter 52 is smaller than an inner diameter of the discharge catheter 51. The injection catheter 52 can be disposed in the lumen 513 of the discharge catheter 51. In addition, the injection catheter 52 is not coupled to the discharge catheter 51, and is movable in the lumen 513 of the discharge catheter 51 along a longitudinal direction D1 (see FIG. 3) of the discharge catheter 51. Since the distal portion of the discharge catheter 51 is opened as the discharge port 511, a distal portion of the injection catheter 52 can pass through the discharge port 511 of the discharge catheter 51 as illustrated in FIG. 3.

As a result, the distal portion of the injection catheter 52 can be exposed from the discharge port 511 of the discharge catheter 51 in the longitudinal direction D1 of the discharge catheter 51. A distance in the longitudinal direction D1 between the distal portion of the discharge catheter 51 and the distal portion of the injection catheter 52 exposed from the discharge port 511 of the discharge catheter 51 can be adjusted to a predetermined distance. The “predetermined distance” in the specification of the present application can be, for example, about 0 cm or more and 30 cm or less (i.e., 0 cm to 30 cm), which makes it possible to avoid a risk that occurs when a catheter is deeply inserted into a subarachnoid space of a patient.

As illustrated in FIG. 2, the distal portion of the injection catheter 52 is opened as the injection port 521, passes through the discharge port 511 of the discharge catheter 51, and is disposed in the subarachnoid space. For example, as indicated by an arrow A13 illustrated in FIG. 2, the injection catheter 52 is inserted into the subarachnoid space of the patient and injects the fluid into cerebrospinal fluid present in the subarachnoid space through the lumen 523 (see FIG. 4) of the injection catheter 52. Note that a force for injecting the fluid into the cerebrospinal fluid is given by the pump 22 as described above with respect to FIG. 1.

In the cutting plane B-B (see FIG. 4) perpendicular to the longitudinal direction D1, a cross-sectional area of the space 53 between the outer side of the injection catheter 52 and the inner side of the discharge catheter 51 is set within a range of a predetermined ratio with respect to a cross-sectional area of the lumen 523 of the injection catheter 52 in order to keep intracranial pressure (ICP) constant within a certain range. Since it is not preferable that the intracranial pressure becomes higher or lower than a limit range, it is preferable to set the ratio of the cross-sectional area of the space 53 with respect to the cross-sectional area of the lumen 523 within a certain range around 1 time (i.e., the ratio of the cross-sectional area of the space 53 to the cross-sectional area of the lumen 523 is 1:1 or equal). The “predetermined ratio” in the specification of the present application can be, for example, preferably about 0.5 times or more and 2 times or less (i.e., the cross-sectional area of the space 53 to the cross-sectional area of the lumen is 0.5:1 to 2:1). However, the “predetermined ratio” in the specification of the present application is not limited to 0.5 times or more and 2 times or less (i.e., 0.5:1 to 2:1).

Next, flows of a fluid generated in a body cavity in fluid circulation will be described with reference to the drawings.

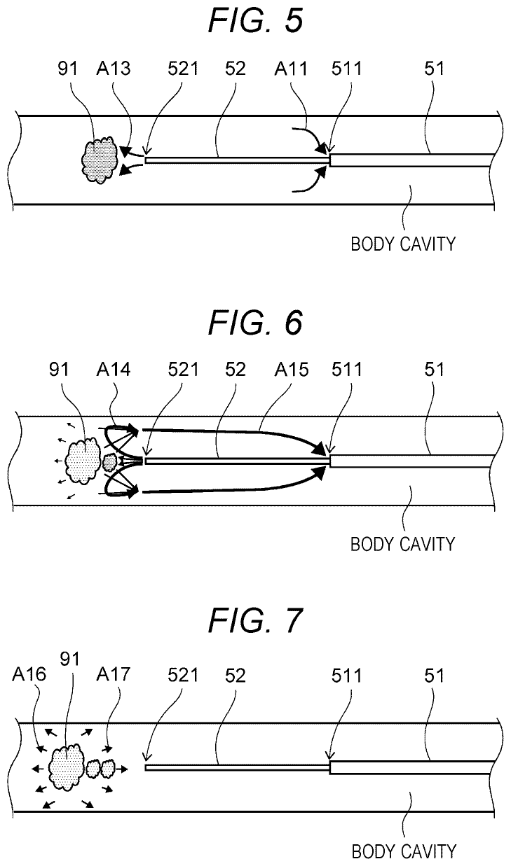

FIG. 5 is a schematic view illustrating a flow of the fluid generated in the body cavity immediately after the start of the fluid circulation.

FIG. 6 is a schematic view illustrating a flow of the fluid generated in the body cavity during the fluid circulation.

FIG. 7 is a schematic view illustrating a flow of the fluid generated in the body cavity after the stop of the fluid circulation.

As illustrated in FIG. 5, immediately after the start of the fluid circulation, a fluid 91 injected into the body cavity from the injection port 521 of the injection catheter 52 flows forward (that is, in a direction in which a brain is present) as indicated by an arrow A13 illustrated in FIG. 5, for example, until a flow of drawing when the cerebrospinal fluid is discharged from the discharge port 511 of the discharge catheter 51 reaches the injection port 521 as will be described later with reference to FIG. 6. A flow rate of the fluid 91 flowing forward is slightly larger than that in a case to be described later with reference to FIG. 6. In addition, in a short time immediately after the start of the fluid circulation, diffusion due to a concentration difference of oxygen or a drug (hereinafter, referred to as “oxygen or the like” for convenience of description) contained in the fluid hardly occurs.

Subsequently, as illustrated in FIG. 6, during the fluid circulation, that is, during continuous circulation of the fluid, the flow of drawing when the cerebrospinal fluid is discharged from the discharge port 511 of the discharge catheter 51 reaches the injection port 521 of the injection catheter 52 to exert an influence. Then, a flow from the injection port 521 of the injection catheter 52 toward the discharge port 511 of the discharge catheter 51 becomes steady. That is, a locally circulating flow in which the fluid 91 injected into the body cavity from the injection port 521 of the injection catheter 52 flows toward the discharge port 511 of the discharge catheter 51 can be generated, for example, as indicated by arrows A14 and A15 illustrated in FIG. 6. As a result, the fluid 91 injected into the body cavity from the injection port 521 of the injection catheter 52 hardly flows forward (that is, in the direction in which the brain is present).

In addition, due to an influence of the locally circulating flow, diffusion of oxygen or the like contained in the fluid 91 to the brain side is decelerated as compared with the time immediately after the start of the fluid circulation. Due to the influence of the locally circulating flow, the fluid 91 diffused to the lumbar side due to the concentration difference of oxygen or the like contained in the fluid 91 is discharged from the discharge port 511 of the discharge catheter 51 out of the body cavity.

As illustrated in FIG. 7, the locally circulating flow generated during the fluid circulation disappears after the stop of the fluid circulation. Therefore, for example, as indicated by arrows A16 and A17 illustrated in FIG. 7, oxygen or the like contained in the fluid 91 is diffused to the brain side and the lumbar side.

As described with respect to FIG. 6, the locally circulating flow in which the fluid 91 injected into the body cavity from the injection port 521 of the injection catheter 52 flows toward the discharge port 511 of the discharge catheter 51 is generated when the continuous circulation of the fluid is executed. As a result, a flow of the fluid 91 from the injection port 521 of the injection catheter 52 toward a treatment area of the brain decreases, and it becomes difficult to efficiently deliver the fluid 91 to the treatment area of the brain.

Therefore, the control unit 21 of the medical system 2 according to the embodiment executes control of setting a first period in which the cerebrospinal fluid is discharged from the body cavity while the fluid 91 is injected into the body cavity and a second period in which the fluid 91 is not injected into the body cavity and the cerebrospinal fluid is not discharged from the body cavity and alternately repeating the first period and the second period. Hereinafter, details of control of the medical system 2 according to the embodiment will be described with reference to the drawings.

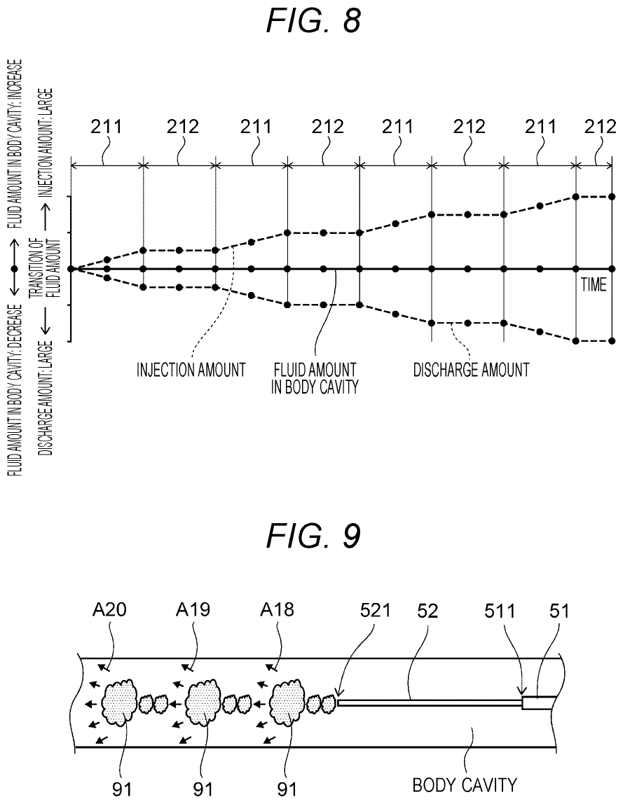

FIG. 8 is a graph showing a relationship among an injection amount, a discharge amount, and a fluid amount in the body cavity under control of the embodiment.

FIG. 9 is a schematic view illustrating a flow of the fluid generated in the body cavity in the control of the embodiment.

The horizontal axis of the graph shown in FIG. 8 represents time. The vertical axis of the graph shown in FIG. 8 represents the transition of the fluid amount. Specifically, the “injection amount” illustrated in FIG. 8 represents an integrated amount of the fluid 91 injected into the body cavity from the start of execution of the control. The “discharge amount” illustrated in FIG. 8 represents an integrated amount of the cerebrospinal fluid discharged out of the body cavity from the start of execution of the control. The “fluid amount in the body cavity” illustrated in FIG. 8 represents a change amount of the cerebrospinal fluid present in the body cavity at the start of execution of the control.

As illustrated in FIG. 8, the control unit 21 of the embodiment sets a first period 211 in which the cerebrospinal fluid is discharged from the body cavity while the fluid 91 is injected into the body cavity, and a second period 212 in which the fluid 91 is not injected into the body cavity and the cerebrospinal fluid is not discharged from the body cavity. That is, in the first period 211, the injection of the fluid 91 into the body cavity and the discharge of the fluid 91 from the body cavity are simultaneously executed. On the other hand, in the second period 212, the injection of the fluid 91 into the body cavity and the discharge of the fluid 91 from the body cavity are simultaneously stopped.

Then, as illustrated in FIG. 8, the control unit 21 alternately repeats the first period 211 and the second period 212. As a result, the injection of the fluid 91 and the discharge of the cerebrospinal fluid are repeated simultaneously and intermittently. The control related to the graph shown in FIG. 8 is an example of “first control” of the disclosure.

In the first control of the medical system 2 according to the embodiment, a state immediately after the start of the fluid circulation described above with reference to FIG. 5 and a state after the stop of the fluid circulation described above with reference to FIG. 7 cyclically exist. Therefore, there is a period in which the locally circulating flow of the fluid 91 injected into the body cavity from the injection port 521 of the injection catheter 52 toward the discharge port 511 of the discharge catheter 51 disappears. As a result, as illustrated in FIG. 9, the amount of the fluid 91 supplied from the injection port 521 of the injection catheter 52 to the brain side increases according to a cycle. In addition, as indicated by arrows A18, A19, and A20 in FIG. 9, the diffusion due to the concentration difference of oxygen or the like contained in the fluid 91 progresses during a period in which there is no locally circulating flow. As a result, the fluid 91 can be efficiently delivered to the treatment area of the brain.

In addition, as illustrated in FIG. 8, the amount of the fluid present in the body cavity at the start of execution of the control is kept in a certain range since the injection of the fluid 91 and the discharge of the cerebrospinal fluid are simultaneously executed, which can suppress fluctuations in intracranial pressure.

In the graph shown in FIG. 8, the injection amount of the fluid 91 in the first period 211 is substantially the same as the discharge amount of the cerebrospinal fluid in the first period 211. Accordingly, the amount of the fluid present in the body cavity at the start of the execution of the control hardly changes with a lapse of time, and is more reliably kept in the certain range, which makes it possible to more reliably suppress the fluctuations in the intracranial pressure.

A cycle from the start of the first period 211 to the end of the second period 212 is substantially constant during the execution of the control. Therefore, movement of the fluid 91 and the diffusion due to the concentration difference of oxygen or the like contained in the fluid 91 stably progress. As a result, a therapeutic substance (oxygen or the like) can be efficiently delivered to the treatment area of the brain. In addition, the amount of the fluid present in the body cavity at the start of execution of the control is stably kept in the certain range, which can suppress fluctuations in intracranial pressure. The duration of the second period 212 is preferably 15 seconds or more. Accordingly, it is possible to secure a sufficient time for the progress of the movement of the fluid 91 and the diffusion due to the concentration difference of oxygen or the like contained in the fluid 91, and the locally circulating flow remaining by inertia is also stopped. As a result, the fluid 91 can be efficiently delivered to the treatment area of the brain.

Next, another control of the medical system according to the embodiment will be described with reference to the drawings.

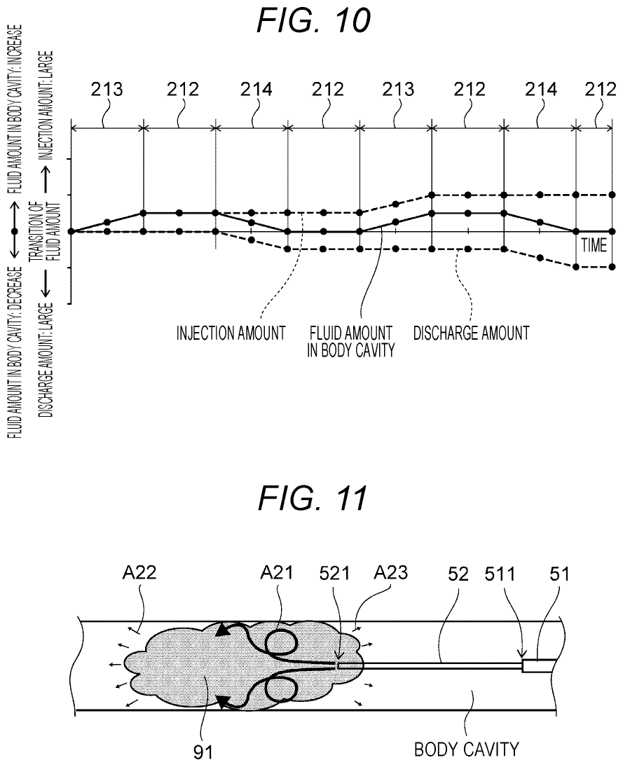

FIG. 10 is a graph showing a relationship among an injection amount, a discharge amount, and a fluid amount in the body cavity under another control of the embodiment.

FIG. 11 is a schematic view illustrating a flow of the fluid generated in the body cavity in another control injection of the embodiment.

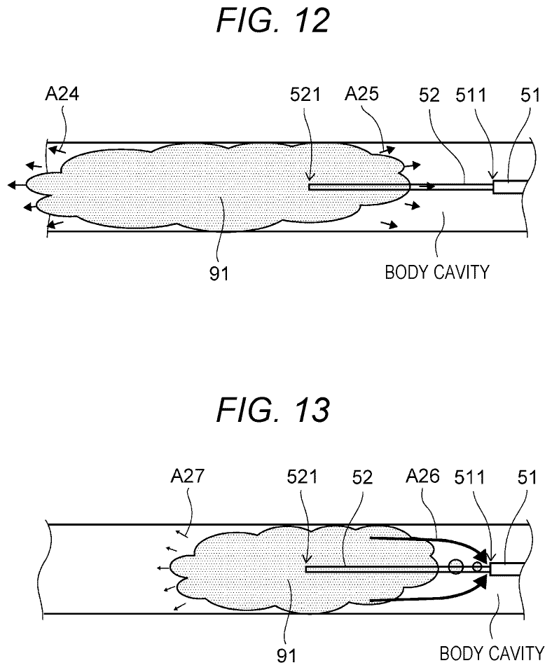

FIG. 12 is a schematic view illustrating a flow of the fluid generated in the body cavity during the stop of another control of the embodiment.

FIG. 13 is a schematic view illustrating a flow of the fluid generated in the body cavity by discharge in another control of the embodiment.

The horizontal and vertical axes of the graph illustrated in FIG. 10 and “injection amount”, “discharge amount”, and “fluid amount in body cavity” illustrated in FIG. 10 are the same as those described above with respect to FIG. 8.

As illustrated in FIG. 10, in another control of the embodiment, the control unit 21 sets a third period 213 in which the fluid 91 is injected into the body cavity and the cerebrospinal fluid is not discharged from the body cavity, and a fourth period 214 in which the fluid 91 is not injected into the body cavity and the cerebrospinal fluid is discharged from the body cavity. That is, in the third period 213, only the injection of the fluid 91 into the body cavity is executed. On the other hand, in the fourth period 214, only the discharge of the fluid 91 from the body cavity is executed.

Then, as illustrated in FIG. 10, the control unit 21 alternately repeats the third period 213 and the fourth period 214. The injection amount of the fluid 91 in the third period 213 is substantially the same as the discharge amount of the cerebrospinal fluid in the fourth period 214. As a result, the injection of the fluid 91 and the discharge of the cerebrospinal fluid are alternately repeated in the same amount as long as an allowable fluctuation range of the intracranial pressure is not exceeded. The control related to the graph shown in FIG. 10 is an example of “second control” of the disclosure.

In the second control of the medical system 2 according to the embodiment, since an injection period of the fluid 91 and a discharge period of the cerebrospinal fluid are set at different times, both the periods are not overlapped, and it is possible to suppress the generation of the locally circulating flow in which the fluid 91 injected into the body cavity from the injection port 521 of the injection catheter 52 flows toward the discharge port 511 of the discharge catheter 51.

As illustrated in FIG. 11, in the third period 213, the fluid 91 injected into the body cavity from the injection port 521 of the injection catheter 52 flows to the brain side according to an injection direction and an injection flow rate. In addition, for example, as indicated by an arrow A21 illustrated in FIG. 11, the fluid 91 and the cerebrospinal fluid are stirred by turbulence generated by resistance between the fluid 91 injected into the body cavity from the injection port 521 of the injection catheter 52 and the cerebrospinal fluid in the body cavity. Furthermore, the diffusion due to the concentration difference of oxygen or the like contained in the fluid 91 progresses, for example, as indicated by arrows A22 and A23 in FIG. 11. As a result, oxygen or the like can be efficiently delivered to the treatment area of the brain. In addition, since the injection amount of the fluid 91 in the third period 213 is substantially the same as the discharge amount of the cerebrospinal fluid in the fourth period 214, the amount of the cerebrospinal fluid in the body cavity is kept in a certain range, which can suppress fluctuations in intracranial pressure.

For example, as indicated by an arrow A26 illustrated in FIG. 13, in the fourth period 214, the same amount of the fluid 91 as the amount of the fluid 91 injected into the body cavity from the injection port 521 of the injection catheter 52 flows toward the discharge port 511 of the discharge catheter 51 and is drawn into the discharge port 511 of the discharge catheter 51. In addition, the fluid 91 injected into the body cavity from the injection port 521 of the injection catheter 52 is pulled back toward the discharge port 511 of the discharge catheter 51 as a whole, and the fluid 91, oxygen, and the like spread in the injection direction remain in the body cavity, for example, as indicated by an arrow A27 illustrated in FIG. 13.

As illustrated in FIG. 10, the control unit 21 further sets the second period 212 (see also FIG. 8) in which the fluid 91 is not injected into the body cavity and the cerebrospinal fluid is not discharged from the body cavity between the third period 213 and the fourth period 214 and between the fourth period 214 and the third period 213. That is, as described above with respect to FIG. 8, the injection of the fluid 91 into the body cavity and the discharge of the fluid 91 from the body cavity are simultaneously stopped in the second period 212. The control unit 21 repeats the third period 213, the second period 212, the fourth period 214, and the second period 212 in this order. In the second period, the diffusion due to the concentration difference of oxygen or the like contained in the fluid 91 progresses toward the brain side and the lumbar side, for example, as indicated by arrows A24 and A25 in FIG. 12, according to the duration of the second period 212 (that is, a stop time).

Furthermore, the control unit 21 selects and executes any one of the first control described above with reference to FIGS. 8 and 9 and the second control described above with reference to FIGS. 10 to 13. Accordingly, the medical system 2 according to the embodiment can flexibly cope with various situations such as the intracranial pressure of the subject and the amount of the cerebrospinal fluid, and can efficiently deliver the fluid 91 to the treatment area of the brain.

Next, an example of an experiment performed by the present inventor will be described with reference to the drawings.

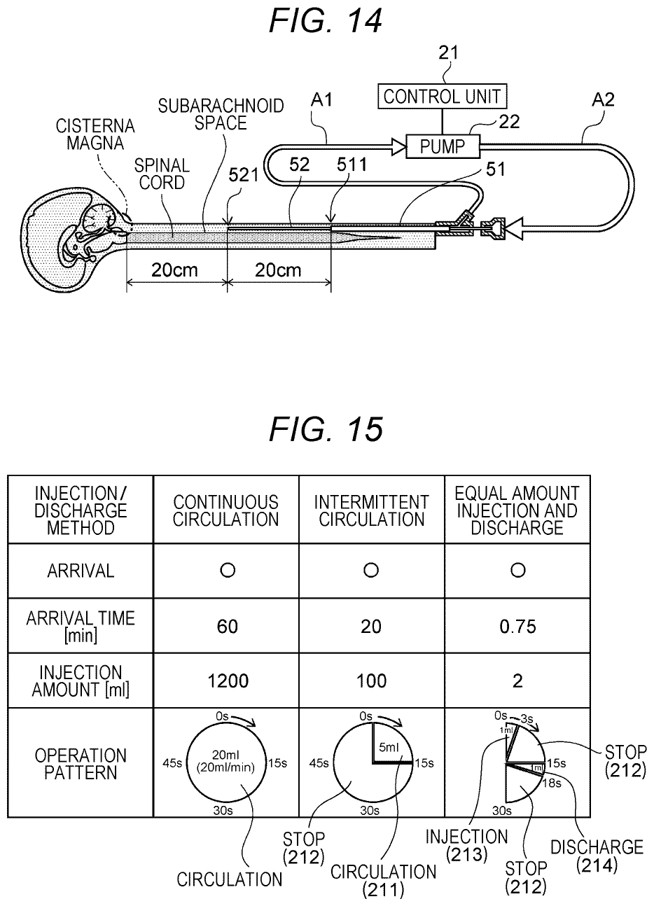

FIG. 14 is a schematic diagram illustrating an outline of the experiment performed by the present inventor.

FIG. 15 is a table showing an example of results of the experiment performed by the present inventors.

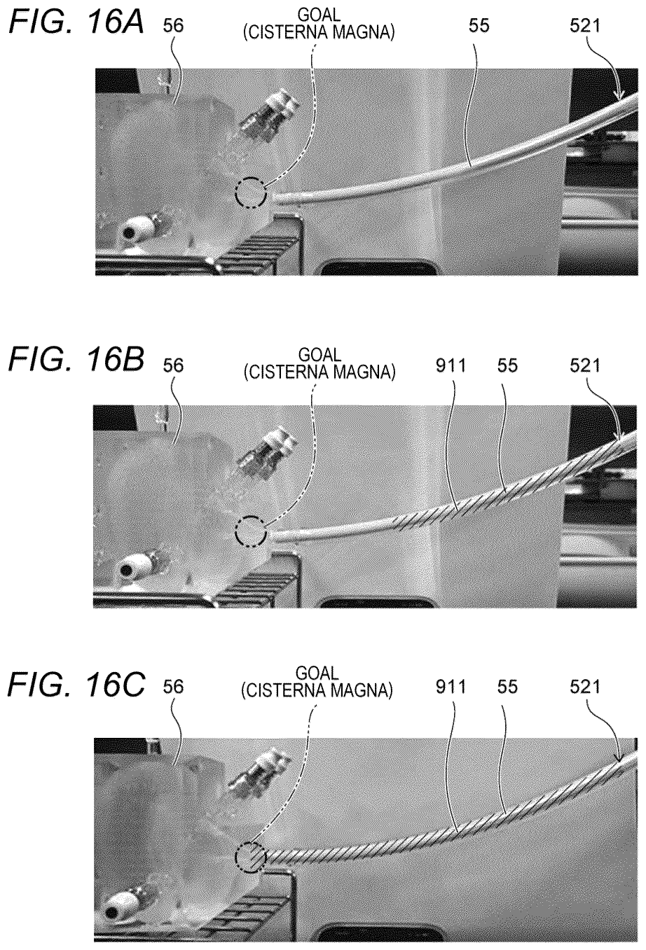

FIGS. 16A to 16C are photographs showing states of the experiment performed by the present inventor.

FIG. 16A is a photograph showing a state at the start of the experiment. FIG. 16B is a photograph showing a state in the middle of the experiment, that is, a state in which colored water 911 arrives at an intermediate position of a tube 55. FIG. 16C is a photograph showing a state in which the colored water 911 arrives at a goal position (or target). Note that the colored water 911 is hatched in the photographs of FIGS. 16B and 16C for convenience of description.

The present inventor injected the colored water 911 into the tube 55 from the injection port 521 of the injection catheter 52 using a model imitating a spinal cord portion of a subarachnoid space as illustrated in FIGS. 16A to 16C, and compared times required from the injection of the colored water 911 into the tube 55 to arrival at a position set as a goal. In the model illustrated in FIGS. 16A to 16C, a lumen of the tube 55 is a portion imitating the subarachnoid space. A lumen of a tank 56 is a portion imitating an intracranial space. The goal position is a position near a connection portion between the tube 55 and the tank 56, and is a position immediately after the colored water 911 having passed through the tube 55 flows into the tank 56. The goal position is a portion imitating cisterna magna.

As illustrated in FIG. 14, the distance in the longitudinal direction D1 (see FIG. 3) between the distal portion (that is, the discharge port 511) of the discharge catheter 51 and the distal portion (that is, the injection port 521) of the injection catheter 52 exposed from the discharge port 511 of the discharge catheter 51 is 20 cm. In addition, a distance in the longitudinal direction D1 between the injection port 521 of the injection catheter 52 and the goal position (that is, the portion imitating the cisterna magna) is 20 cm.

An injection and discharge method in the experiment will be described with reference to FIG. 15.

“Continuous circulation” in the table shown in FIG. 15 is a method of continuously injecting and discharging the colored water 911, and is a method as a comparative example. That is, as in the table shown in FIG. 15, in the “continuous circulation”, the control unit 21 sets a period for continuously injecting and discharging the colored water 911 at a flow rate of 20 mL/min.

“Intermittent circulation” in the table shown in FIG. 15 is an injection and discharge method when the first control described above with reference to FIGS. 8 and 9 is executed. That is, as in the table shown in FIG. 15, in the “intermittent circulation”, the control unit 21 sets the first period 211 in which the colored water 911 is discharged from the tube 55 at a flow rate of 20 mL/min while the colored water 911 is injected into the tube 55 at a flow rate of 20 mL/min, and the second period 212 in which the colored water 911 is not injected into the tube 55 and the colored water 911 is not discharged from the tube 55. The duration of the first period 211 is 15 seconds. In the first period 211, the colored water 911 at the flow rate of 20 mL/min is injected into the tube 55 for 15 seconds, so that an injection amount of the colored water 911 is 5 mL. In addition, similarly, the amount discharged from the tube 55 is also 5 mL. The duration of the second period 212 is 45 seconds. Then, the control unit 21 alternately repeats the first period 211 and the second period 212.

In the table shown in FIG. 15, “equal amount injection and discharge” is an injection and discharge method when the second control described above with reference to FIGS. 10 to 13 is executed. That is, as in the table shown in FIG. 15, in the “equal amount injection and discharge”, the control unit 21 sets the third period 213 in which the colored water 911 is injected into the tube 55 and the colored water 911 is not discharged from the tube 55, and the fourth period 214 in which the colored water 911 is not injected into the tube 55 and the colored water 911 is discharged from the tube 55. The duration of the third period 213 is 3 seconds. In the third period 213, the colored water 911 at a flow rate of 20 mL/min is injected into the tube 55 for 3 seconds, so that an injection amount of the colored water 911 is 1 mL. The duration of the fourth period 214 is 3 seconds. In the fourth period 214, the colored water 911 at a flow rate of 20 mL/min is discharged from the tube 55 for 3 seconds, so that a discharge amount of the colored water 911 is 1 mL. Then, the control unit 21 alternately repeats the third period 213 and the fourth period 214. Furthermore, in the “equal amount injection and discharge”, the control unit 21 sets the second period 212 in which the colored water 911 is not injected into the tube 55 and the colored water 911 is not discharged from the tube 55 between the third period 213 and the fourth period 214 and between the fourth period 214 and the third period 213. The duration of the second period 212 is 12 seconds. In this manner, in the “equal amount injection and discharge”, the control unit 21 repeats the third period 213, the second period 212, the fourth period 214, and the second period 212 in this order.

An example of results of the experiment is shown in the table of FIG. 15. That is, the colored water 911 could arrive at the goal position in all of the “continuous circulation”, the “intermittent circulation”, and the “equal amount injection and discharge”. A time required for the colored water 911 to arrive at the goal position after being injected into the tube 55 is 60 minutes in the “continuous circulation”, 20 minutes in the “intermittent circulation”, and 0.75 minutes (or 45 seconds) in the “equal amount injection and discharge”. The amount of the colored water 911 injected until the colored water 911 arrives at the goal position after being injected into the tube 55 is 1200 mL in the “continuous circulation”, 100 mL in the “intermittent circulation”, and 2 mL in the “equal amount injection and discharge”. From the results of the experiment, it can be seen that the “intermittent circulation” and the “equal amount injection and discharge” can efficiently deliver the colored water 911 to the goal position as compared with the “continuous circulation”.

Next, a fluid circulation system according to the embodiment will be described with reference to the drawings.

Note that, in a case where components of a fluid circulation system 3 are similar to the components of the medical system 2 described above with reference to FIGS. 1 to 16C, redundant description will be appropriately omitted, and differences will be mainly described hereinafter.

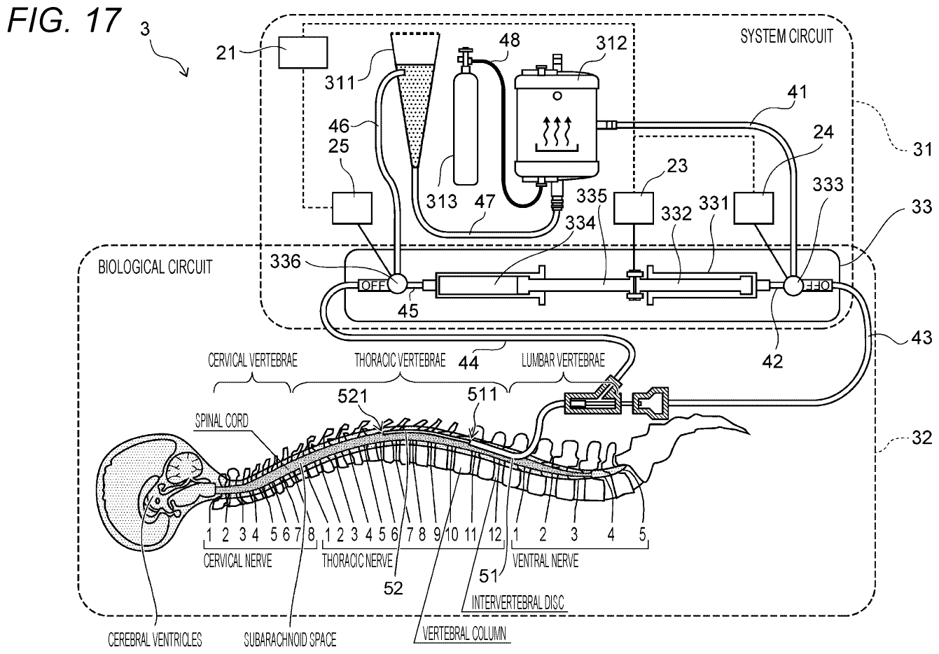

FIG. 17 is a schematic diagram illustrating an outline of the fluid circulation system according to the embodiment.

The fluid circulation system 3 according to the embodiment injects a fluid into a body and discharges the fluid out of the body to circulate the fluid. Examples of the inside of the body include the inside of a body cavity in which cerebrospinal fluid of a subject is present. In the description of the fluid circulation system 3 according to the embodiment, a case where the fluid to be injected into the body is a high oxygen solution will be described as an example.

As illustrated in FIG. 17, the fluid circulation system 3 includes a system circuit 31, a biological circuit 32, and a pump unit 33.

The system circuit 31 is a portion that generates the high oxygen solution, adjusts a temperature, and adjusts a total volume of the fluid in a circulation circuit, and includes the control unit 21, a reservoir 311, an oxygenation mechanism 312, and an oxygen supply source 313. The oxygenation mechanism 312 of the embodiment is an example of an “oxygenator” of the disclosure.

The system circuit 31 further includes a first drive unit 23, a second drive unit 24, and a third drive unit 25. The first drive unit 23 of the embodiment is an example of a “piston drive unit” of the disclosure. Each of the first drive unit 23, the second drive unit 24, and the third drive unit 25 is connected to the control unit 21, and operates on the basis of a control signal transmitted from control unit 21. Examples of the first drive unit 23, the second drive unit 24, and the third drive unit 25 include actuators such as motors. Note that the first drive unit 23, the second drive unit 24, and the third drive unit 25 may be provided in the pump unit 33.

The biological circuit 32 is a portion that injects the fluid into the body and discharges the fluid out of the body, and includes the discharge catheter 51 and the injection catheter 52. The discharge catheter 51 and the injection catheter 52 are connected to the system circuit 31 via the pump unit 33. The discharge catheter 51 and the injection catheter 52 are the same as those described with respect to FIGS. 1 to 16C.

The pump unit 33 functions as a fluid delivery unit shared by the system circuit 31 and the biological circuit 32. The pump unit 33 includes a first syringe 331, a first piston 332, a first flow path switching unit 333, a second syringe 334, a second piston 335, and a second flow path switching unit 336. As described above, the pump unit 33 may include the first drive unit 23, the second drive unit 24, and the third drive unit 25.

The first syringe 331 of the embodiment is an example of an “injection fluid storage unit” of the disclosure. The first piston 332 of the embodiment is an example of a “first fluid delivery unit” of the disclosure. The second syringe 334 of the embodiment is an example of a “discharge fluid storage unit” of the disclosure. The second piston 335 of the embodiment is an example of a “second fluid delivery unit” of the disclosure.

As illustrated in FIG. 17, the oxygenation mechanism 312 is connected to the first flow path switching unit 333 via a first tube 41. In addition, the oxygenation mechanism 312 is connected to the reservoir 311 via a seventh tube 47. Furthermore, the oxygenation mechanism 312 is connected to the oxygen supply source 313 via an eighth tube 48. The oxygenation mechanism 312 mixes oxygen supplied from the oxygen supply source 313 through the eighth tube with cerebrospinal fluid supplied from the reservoir 311 through the seventh tube 47 or artificial cerebrospinal fluid such as lactated Ringer's solution or a mixed solution of the cerebrospinal fluid and the artificial cerebrospinal fluid to generate oxygenated cerebrospinal fluid. In addition, the oxygenation mechanism 312 also includes a heat exchanger that adjusts a temperature of the oxygenated cerebrospinal fluid. Then, the oxygenation mechanism 312 supplies the oxygenated cerebrospinal fluid as the high oxygen solution to the first flow path switching unit 333 through the first tube 41. As the oxygenation mechanism 312 in the embodiment, a hollow fiber oxygenator for adding oxygen to blood can be used.

The first flow path switching unit 333 is provided at a connection portion among the first tube 41, a second tube 42, and a third tube 43, and can switch between a flow path in which the first tube 41 and the second tube 42 are connected and a flow path in which the second tube 42 and the third tube 43 are connected. Specifically, the second drive unit 24 controls an operation of the first flow path switching unit 333 on the basis of a control signal transmitted from the control unit 21 to switch between the flow path in which the first tube 41 and the second tube 42 are connected and the flow path in which the second tube 42 and the third tube 43 are connected.

The first flow path switching unit 333 is connected to the first syringe 331 via the second tube 42. The first piston 332 can slide and reciprocate in the first syringe 331. Specifically, the first drive unit 23 controls an operation of the first piston 332 on the basis of a control signal transmitted from the control unit 21 to reciprocate the first piston 332 in the first syringe 331. When the first piston 332 moves in a direction to be removed from the first syringe 331 in a state where the first flow path switching unit 333 connects the first tube 41 and the second tube 42, the first syringe 331 stores the fluid supplied from the oxygenation mechanism 312 through the first tube 41 and the second tube 42. On the other hand, when the first piston 332 moves in a direction to be inserted into the first syringe 331 in a state where the first flow path switching unit 333 connects the second tube 42 and the third tube 43, the first piston 332 supplies the fluid stored in the first syringe 331 to the injection catheter 52 through the second tube 42 and the third tube 43.

In this manner, the first syringe 331 temporarily stores the fluid to be injected into the body. In addition, the first piston 332 is connected to the first syringe 331 and connected to the injection catheter 52 via the second tube 42 and the third tube 43, and supplies the fluid to the first syringe 331 and supplies the fluid stored in the first syringe 331 to the injection catheter 52.

As illustrated in FIG. 17, the reservoir 311 is connected to the second flow path switching unit 336 via a sixth tube 46. In addition, the reservoir 311 is connected to the oxygenation mechanism 312 via the seventh tube 47. The reservoir 311 temporarily stores the cerebrospinal fluid supplied through the sixth tube 46. Then, the reservoir 311 supplies the stored cerebrospinal fluid to the oxygenation mechanism 312 through the seventh tube 47. The reservoir 311 has a structure in which the inside and the outside communicate with each other, and can extract gas contained in the stored cerebrospinal fluid to the outside. That is, the reservoir 311 has a function as an air trap.

The second flow path switching unit 336 is provided at a connection portion among a fourth tube 44, a fifth tube 45, and the sixth tube 46, and can switch between a flow path in which the fourth tube 44 and the fifth tube 45 are connected and a flow path in which the fifth tube 45 and the sixth tube 46 are connected. Specifically, the third drive unit 25 controls an operation of the second flow path switching unit 336 on the basis of a control signal transmitted from the control unit 21 to switch between the flow path in which the fourth tube 44 and the fifth tube 45 are connected and the flow path in which the fifth tube 45 and the sixth tube 46 are connected.

The second flow path switching unit 336 is connected to the second syringe 334 via the fifth tube 45. The second piston 335 can slide and reciprocate in the second syringe 334. Specifically, the first drive unit 23 controls an operation of the second piston 335 on the basis of a control signal transmitted from the control unit 21 to reciprocate the second piston 335 in the second syringe 334. That is, as illustrated in FIG. 17, the first piston 332 and the second piston 335 are connected to each other, and reciprocate by receiving a driving force of the first drive unit 23 in a state of being connected to each other. When the second piston 335 moves in a direction to be removed from the second syringe 334 in a state where the second flow path switching unit 336 connects the fourth tube 44 and the fifth tube 45, the second syringe 334 stores the fluid (that is, cerebrospinal fluid) discharged from the inside of the body to the outside of the body through the fourth tube 44 and the fifth tube 45. On the other hand, when the second piston 335 moves in a direction to be inserted into the second syringe 334 in a state where the second flow path switching unit 336 connects the fifth tube 45 and the sixth tube 46, the second piston 335 supplies the fluid stored in the second syringe 334 to the reservoir 311 through the fifth tube 45 and the sixth tube 46.

In this manner, the second piston 335 is connected to the second syringe 334 and connected to the discharge catheter 51 via the fourth tube 44 and the fifth tube 45, and supplies the fluid to the second syringe 334 and supplies the fluid stored in the second syringe 334 to the reservoir 311.

The first syringe 331 and the second syringe 334 are formed in the same shape and volume. In addition, as described above, the first piston 332 and the second piston 335 are connected to each other, and reciprocate by receiving the driving force of the first drive unit 23 in the state of being connected to each other. As a result, a volume of the fluid injected into the body is always equal to a volume of the fluid discharged out of the body.

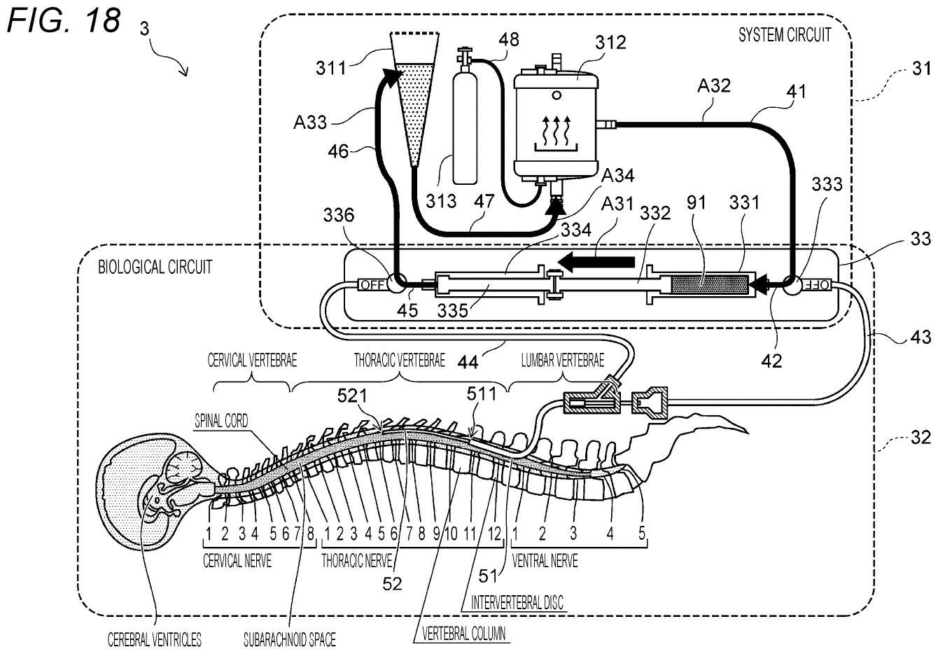

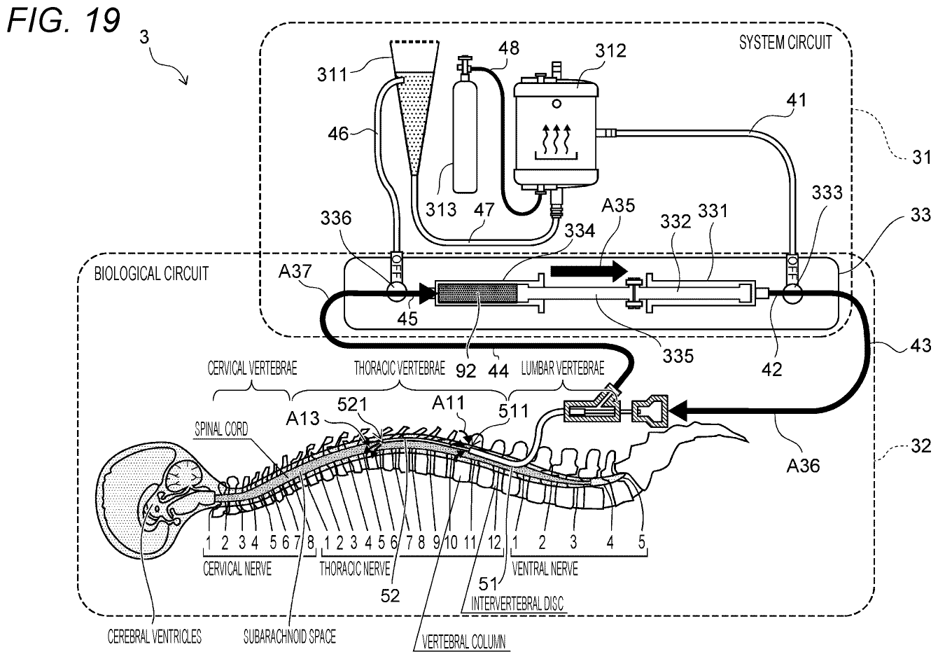

FIGS. 18 and 19 are schematic diagrams illustrating an operation of the fluid circulation system according to the embodiment.

The operation of the fluid circulation system 3 described with reference to FIGS. 18 and 19 is the operation based on the first control described above with reference to FIGS. 8 and 9.

Note that the control unit 21, the first drive unit 23, the second drive unit 24, and the third drive unit 25 are omitted in FIGS. 18 and 19 for convenience of description. In addition, the following operation will be described on the assumption that the tubes, the reservoir, the respective pumps (syringes), and the oxygenation mechanism in the circuit start from a primed state filled with artificial cerebrospinal fluid such as lactated Ringer's solution in advance.

First, as illustrated in FIG. 18, as a first step, the control unit 21 transmits a control signal to the second drive unit 24 to control the operation of the first flow path switching unit 333 and connect the first tube 41 and the second tube 42. In addition, the control unit 21 transmits a control signal to the third drive unit 25 to control the operation of the second flow path switching unit 336 and connect the fifth tube 45 and the sixth tube 46. In this state, the control unit 21 transmits a control signal to the first drive unit 23 to move the first piston 332 in the removal direction of the first syringe 331 and move the second piston 335 in the insertion direction of the second syringe 334 as indicated by an arrow A31 illustrated in FIG. 18.

Then, as indicated by an arrow A32 illustrated in FIG. 18, the fluid 91 (that is, the high oxygen solution) generated in the oxygenation mechanism 312 is supplied to and stored in the first syringe 331 through the first tube 41 and the second tube 42. In addition, as indicated by an arrow A33 illustrated in FIG. 18, cerebrospinal fluid 92 (see FIG. 19) stored in the second syringe 334 is supplied to and stored in the reservoir 311 through the fifth tube 45 and the sixth tube 46. Furthermore, as indicated by an arrow A34 illustrated in FIG. 18, the cerebrospinal fluid 92 stored in the reservoir 311 is supplied to the oxygenation mechanism 312 through the seventh tube 47.

Subsequently, as illustrated in FIG. 19, as a second step, the control unit 21 transmits a control signal to the second drive unit 24 to control the operation of the first flow path switching unit 333 and connect the second tube 42 and the third tube 43. In addition, the control unit 21 transmits a control signal to the third drive unit 25 to control the operation of the second flow path switching unit 336 and connect the fourth tube 44 and the fifth tube 45. In this state, the control unit 21 transmits a control signal to the first drive unit 23 to move the first piston 332 in the insertion direction of the first syringe 331 and move the second piston 335 in the removal direction of the second syringe 334 as indicated by an arrow A35 illustrated in FIG. 19.

Then, as indicated by an arrow A36 illustrated in FIG. 19, the fluid 91 (that is, the high oxygen solution) stored in the first syringe 331 is supplied to the injection catheter 52 through the second tube 42 and the third tube 43. Then, for example, as indicated by an arrow A13 illustrated in FIG. 19, the fluid 91 is injected into the body from the injection port 521 of the injection catheter 52. On the other hand, for example, as indicated by an arrow A11 illustrated in FIG. 19, the cerebrospinal fluid 92 in the body is drawn into the discharge port 511 of the discharge catheter 51. Then, as indicated by an arrow A37 illustrated in FIG. 19, the cerebrospinal fluid 92 is drawn and stored in the second syringe 334 through the fourth tube 44 and the fifth tube 45.

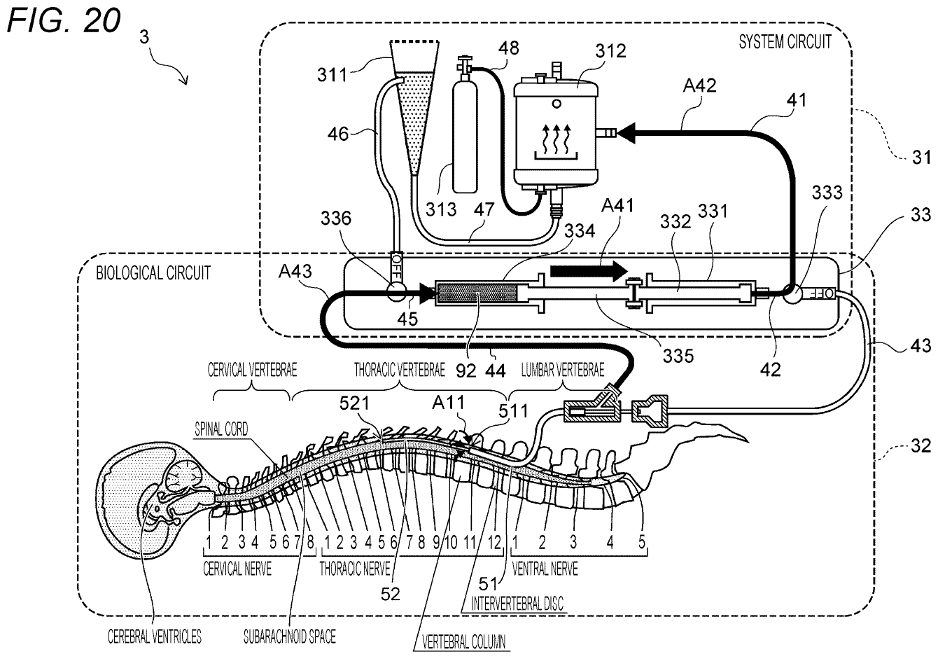

FIGS. 20 to 23 are schematic diagrams illustrating another operation of the fluid circulation system according to the embodiment.

The operation of the fluid circulation system 3 described with reference to FIGS. 20 to 23 is the operation based on the second control described above with reference to FIGS. 10 to 13.

Note that the control unit 21, the first drive unit 23, the second drive unit 24, and the third drive unit 25 are omitted in FIGS. 20 to 23 for convenience of description.

First, as illustrated in FIG. 20, as a first step, the control unit 21 transmits a control signal to the second drive unit 24 to control the operation of the first flow path switching unit 333 and connect the first tube 41 and the second tube 42. In addition, the control unit 21 transmits a control signal to the third drive unit 25 to control the operation of the second flow path switching unit 336 and connect the fourth tube 44 and the fifth tube 45. In this state, the control unit 21 transmits a control signal to the first drive unit 23 to move the first piston 332 in the insertion direction of the first syringe 331 and move the second piston 335 in the removal direction of the second syringe 334 as indicated by an arrow A41 illustrated in FIG. 20.

Then, as indicated by an arrow A42 illustrated in FIG. 20, the fluid 91 (that is, the high oxygen solution) stored in the first syringe 331 is returned to the oxygenation mechanism 312 through the first tube 41 and the second tube 42. On the other hand, for example, as indicated by an arrow A11 illustrated in FIG. 20, the cerebrospinal fluid 92 in the body is drawn into the discharge port 511 of the discharge catheter 51. Then, as indicated by an arrow A43 illustrated in FIG. 20, the cerebrospinal fluid 92 is drawn and stored in the second syringe 334 through the fourth tube 44 and the fifth tube 45.

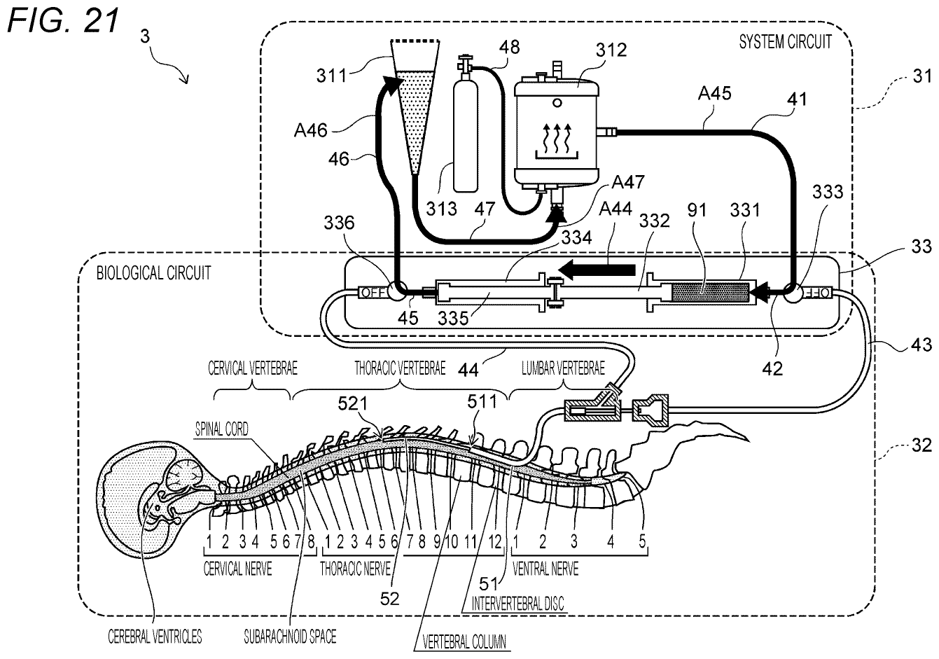

Subsequently, as illustrated in FIG. 21, as a second step, the control unit 21 transmits a control signal to the second drive unit 24 to control the operation of the first flow path switching unit 333 and connect the first tube 41 and the second tube 42 (that is, maintain the connection state). In addition, the control unit 21 transmits a control signal to the third drive unit 25 to control the operation of the second flow path switching unit 336 and connect the fifth tube 45 and the sixth tube 46. In this state, the control unit 21 transmits a control signal to the first drive unit 23 to move the first piston 332 in the removal direction of the first syringe 331 and move the second piston 335 in the insertion direction of the second syringe 334 as indicated by an arrow A44 illustrated in FIG. 21.

Then, as indicated by an arrow A45 illustrated in FIG. 21, the fluid 91 (that is, the high oxygen solution) generated in the oxygenation mechanism 312 is supplied to and stored in the first syringe 331 through the first tube 41 and the second tube 42. In addition, as indicated by an arrow A46 illustrated in FIG. 21, the cerebrospinal fluid 92 stored in the second syringe 334 is supplied to and stored in the reservoir 311 through the fifth tube 45 and the sixth tube 46. Furthermore, as indicated by an arrow A47 illustrated in FIG. 21, the cerebrospinal fluid 92 stored in the reservoir 311 is supplied to the oxygenation mechanism 312 through the seventh tube 47.

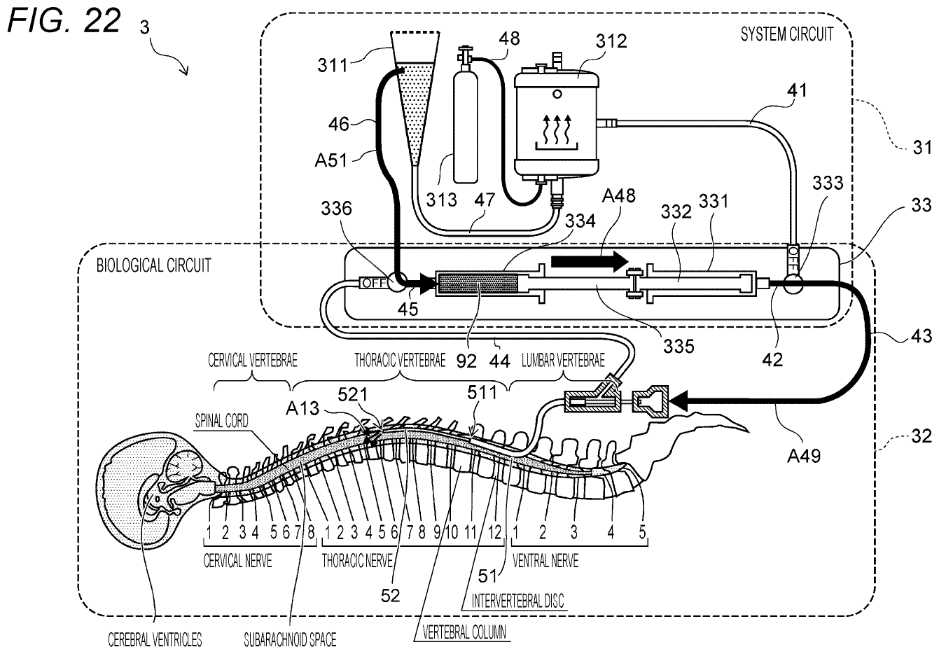

Subsequently, as illustrated in FIG. 22, as a third step, the control unit 21 transmits a control signal to the second drive unit 24 to control the operation of the first flow path switching unit 333 and connect the second tube 42 and the third tube 43. In addition, the control unit 21 transmits a control signal to the third drive unit 25 to control the operation of the second flow path switching unit 336 and connect fifth tube 45 and sixth tube 46 (that is, maintain the connection state). In this state, the control unit 21 transmits a control signal to the first drive unit 23 to move the first piston 332 in the insertion direction of the first syringe 331 and move the second piston 335 in the removal direction of the second syringe 334 as indicated by an arrow A48 illustrated in FIG. 22.

Then, as indicated by an arrow A49 illustrated in FIG. 22, the fluid 91 (that is, the high oxygen solution) stored in the first syringe 331 is supplied to the injection catheter 52 through the second tube 42 and the third tube 43. Then, for example, as indicated by an arrow A13 illustrated in FIG. 22, the fluid 91 is injected into the body from the injection port 521 of the injection catheter 52. On the other hand, as indicated by an arrow A51 illustrated in FIG. 22, the cerebrospinal fluid 92 stored in the reservoir 311 is returned to the second syringe 334 through the fifth tube 45 and the sixth tube 46.

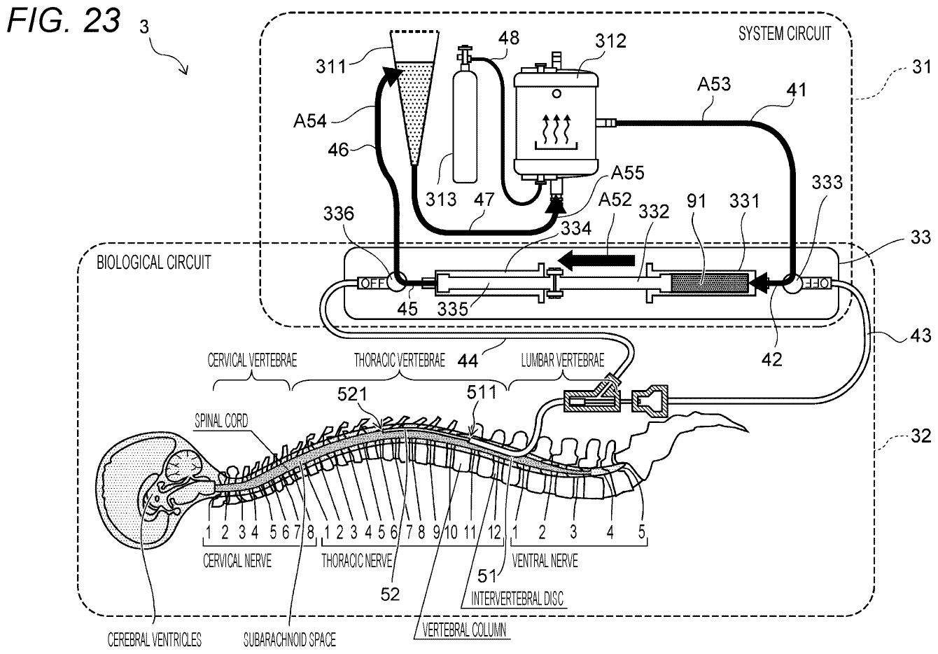

Subsequently, as illustrated in FIG. 23, as a fourth step, the control unit 21 transmits a control signal to the second drive unit 24 to control the operation of the first flow path switching unit 333 and connects the first tube 41 and the second tube 42. In addition, the control unit 21 transmits a control signal to the third drive unit 25 to control the operation of the second flow path switching unit 336 and connect fifth tube 45 and sixth tube 46 (that is, maintain the connection state). In this state, the control unit 21 transmits a control signal to the first drive unit 23 to move the first piston 332 in the removal direction of the first syringe 331 and move the second piston 335 in the insertion direction of the second syringe 334 as indicated by an arrow A52 illustrated in FIG. 23.

Then, as indicated by an arrow A53 illustrated in FIG. 23, the fluid 91 (that is, the high oxygen solution) generated in the oxygenation mechanism 312 is supplied to and stored in the first syringe 331 through the first tube 41 and the second tube 42. In addition, as indicated by an arrow A54 illustrated in FIG. 23, the cerebrospinal fluid 92 stored in the second syringe 334 is supplied to and stored in the reservoir 311 through the fifth tube 45 and the sixth tube 46. Furthermore, as indicated by an arrow A55 illustrated in FIG. 23, the cerebrospinal fluid 92 stored in the reservoir 311 is supplied to the oxygenation mechanism 312 through the seventh tube 47.

According to the fluid circulation system 3 of the embodiment, the circuit for circulating the fluid is divided into a circuit (that is, the biological circuit 32) including the injection catheter 52 and the discharge catheter 51 and the system circuit 31 connected to the injection catheter 52 and the discharge catheter 51. In addition, the first piston 332 that supplies the fluid to the injection catheter 52 and the second piston 335 that supplies the fluid to the system circuit 31 function as the fluid delivery unit shared by the biological circuit 32 and the system circuit 31. In addition, the first flow path switching unit 333 and the second flow path switching unit 336 can switch the flow paths in which the fluid flows. Since the fluid circulation system 3 has such a configuration, the system circuit 31 can cope with an increase in the total volume of the fluid caused by the cavitation phenomenon and a decrease in the total volume of the fluid caused by evaporation while maintaining a situation on a living body side, and can suppress the increase and decrease in the total volume of the fluid, which can suppress fluctuations in intracranial pressure.

In addition, the first piston 332 and the second piston 335 operate in conjunction with each other, and function as one fluid delivery unit shared by the biological circuit 32 and the system circuit 31. The first syringe 331 and the second syringe 334 are formed in the same shape and volume. Therefore, the volume of the fluid injected into the body is always equal to the volume of the fluid discharged out of the body. In such a fixed displacement pump, the amount of the fluid injected and discharged in one cycle does not fluctuate, and an injection amount and a discharge amount can be reliably made identical as compared with a variable displacement pump. Therefore, the amount of cerebrospinal fluid in the body can be more reliably kept in a certain range. This makes it possible to more reliably suppress the fluctuations in the intracranial pressure.

Furthermore, in a case where the fluid to be injected into the body is a high oxygen solution, the high oxygen solution can be efficiently delivered to a treatment area of a brain while suppressing the fluctuations in the intracranial pressure.

The detailed description above describes embodiments of a medical system and a fluid circulation system which are used for treatment of a brain disease. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical system configured to inject a fluid into a body cavity in which cerebrospinal fluid of a subject is present and to discharge the cerebrospinal fluid present in the body cavity from the body cavity, the medical system comprising:
a control unit configured to execute control of:
setting a first period in which the cerebrospinal fluid is discharged from the body cavity while the fluid is injected into the body cavity and a second period in which the fluid is not injected into the body cavity and the cerebrospinal fluid is not discharged from the body cavity; and
alternately repeating the first period and the second period.

2. The medical system according to claim 1, wherein
an injection amount of the fluid in the first period is substantially identical to a discharge amount of the cerebrospinal fluid in the first period.

3. The medical system according to claim 1, wherein
a cycle from a start of the first period to an end of the second period is substantially constant.

4. The medical system according to claim 1, wherein
a duration of the second period is 15 seconds or more.

5. The medical system according to claim 1, wherein
the control is a first control; and
the control unit is further configured to execute a second control of:
setting a third period in which the fluid is injected into the body cavity and the cerebrospinal fluid is not discharged from the body cavity and a fourth period in which the fluid is not injected into the body cavity and the cerebrospinal fluid is discharged from the body cavity; and
alternately repeating the third period and the fourth period, and setting an injection amount of the fluid in the third period to be substantially identical to a discharge amount of the cerebrospinal fluid in the fourth period, and selecting and executing one or more of the first control and the second control.

6. The medical system according to claim 1, further comprising:
an injection catheter configured to inject the fluid into the body cavity;
a discharge catheter configured to discharge the cerebrospinal fluid from the body cavity; and
wherein a distance between a distal end of the injection catheter and a distal end of the discharge catheter is 30 cm or less at a start of execution of the control.

7. The medical system according to claim 6, wherein a distal portion of the discharge catheter is open as a discharge port and configured to be disposed in a subarachnoid space of the subject, and wherein the discharge catheter is configured to draw the cerebrospinal fluid present in the subarachnoid space in a vicinity of lumbar vertebrae into a space between a lumen of the discharge catheter and an outer surface of the injection catheter through the discharge port.

8. The medical system according to claim 7, wherein an outer diameter of the injection catheter is smaller than an inner diameter of the discharge catheter, and wherein the injection catheter is disposed in the lumen of the discharge catheter, and the injection catheter is configured to be movable in the lumen of the discharge catheter along a longitudinal direction of the discharge catheter.

9. The medical system according to claim 7, wherein a cross-sectional area of a space between an outer side of the injection catheter and an inner side of the discharge catheter is set within a range of a predetermined ratio with respect to a cross-sectional area of the lumen of the injection catheter in order to keep intracranial pressure constant within a certain range, and wherein predetermined ratio of the cross-sectional area of the space to the cross-sectional area of the lumen is 0.5:1 to 2:1.

10. A fluid circulation system configured to inject a fluid into a body and discharge the fluid out of the body to circulate the fluid, the fluid circulation system comprising:
an injection catheter configured to inject the fluid into the body;
a discharge catheter configured to discharge the fluid from an inside of the body to an outside of the body;
a system circuit connected to the injection catheter and the discharge catheter;
an injection fluid storage unit configured to store the fluid supplied from the system circuit and injected into the body;
a discharge fluid storage unit configured to store the fluid discharged from the inside of the body to the outside of the body;
a first fluid delivery unit that is connected to the injection fluid storage unit and the injection catheter, the first fluid delivery unit configured to supply the fluid to the injection fluid storage unit, and supply the fluid stored in the injection fluid storage unit to the injection catheter;
a second fluid delivery unit that is connected to the discharge fluid storage unit and the discharge catheter, the second fluid delivery unit configured to supply the fluid to the discharge fluid storage unit, and supply the fluid stored in the discharge fluid storage unit to the system circuit;
a first flow path switching unit that is provided on a downstream side of the injection fluid storage unit and is configured to switch between a flow path in which the system circuit is connected to the injection fluid storage unit and a flow path in which the injection fluid storage unit is connected to the injection catheter; and
a second flow path switching unit that is provided on an upstream side of the discharge fluid storage unit and is configured to switch between a flow path in which the discharge catheter is connected to the discharge fluid storage unit and a flow path in which the discharge fluid storage unit is connected to the system circuit.

11. The fluid circulation system according to claim 10, wherein
as the first fluid delivery unit and the second fluid delivery unit operate in conjunction with each other, a volume of the fluid injected into the body is always equal to a volume of the fluid discharged out of the body.

12. The fluid circulation system according to claim 10, further comprising
a piston drive unit;
wherein the injection fluid storage unit is a first syringe configured to store the fluid;
the discharge fluid storage unit is a second syringe configured to store the fluid;
the first fluid delivery unit is a first piston configured to slide and reciprocate in the first syringe;
the second fluid delivery unit is a second piston configured to slide and reciprocate in the second syringe; and
the first piston and the second piston are connected to each other, and are configured to reciprocate by the piston drive unit in a state of being connected to each other.

13. The fluid circulation system according to claim 12, wherein
as the first syringe and the second syringe are formed to have identical shape and volume, a volume of the fluid injected into the body is always equal to a volume of the fluid discharged out of the body.

14. The fluid circulation system according to claim 10, wherein
the system circuit includes an oxygenator configured to add oxygen to the fluid discharged out of the body.

15. A method for injecting a fluid into a body cavity in which cerebrospinal fluid of a subject is present and to discharge the cerebrospinal fluid present in the body cavity from the body cavity, the method comprising:
setting a first period in which the cerebrospinal fluid is discharged from the body cavity while the fluid is injected into the body cavity and a second period in which the fluid is not injected into the body cavity and the cerebrospinal fluid is not discharged from the body cavity; and
alternately repeating the first period and the second period.

16. The method according to claim 15, further comprising:
injecting an amount of the fluid in the first period that is substantially identical to a discharge amount of the cerebrospinal fluid in the first period.

17. The method according to claim 15, wherein
a cycle from a start of the first period to an end of the second period is substantially constant.

18. The method according to claim 15, wherein a duration of the second period is 15 seconds or more.

19. The method according to claim 15, wherein the control is a first control, and the method further comprises:
executing a second control of:
setting a third period in which the fluid is injected into the body cavity and the cerebrospinal fluid is not discharged from the body cavity and a fourth period in which the fluid is not injected into the body cavity and the cerebrospinal fluid is discharged from the body cavity; and
alternately repeating the third period and the fourth period, and setting an injection amount of the fluid in the third period to be substantially identical to a discharge amount of the cerebrospinal fluid in the fourth period, and selecting and executing one or more of the first control and the second control.

20. The method according to claim 15, further comprising:
injecting the fluid into the body cavity with an injection catheter;
discharging the cerebrospinal fluid from the body cavity with a discharge catheter; and
wherein a distance between a distal end of the injection catheter and a distal end of the discharge catheter is 30 cm or less at a start of execution of the method.
